(19) United States
(12) Patent Application Publication
(10) Pub. No.: US 2022/0079511 A1
(43) Pub. Date: Mar. 17, 2022

(54) MEASUREMENT OF NEUROMOTOR COORDINATION FROM SPEECH

(71) Applicants: Massachusetts Institute of Technology, Cambridge, MA (US); University of Maryland, College Park, MD (US)

(72) Inventors: Thomas Quatieri, Newtonville, MA (US); Adam Lammert, Lexington, MA (US); Carol Epsy-Wilson, Silver Spring, MD (US)

(21) Appl. No.: 17/021,230

(22) Filed: Sep. 15, 2020

(51) Int. Cl. A61B 5/00; G10L 25/66; G10L 25/30; G10L 25/21; G10L 25/24; G06T 13/80

(52) U.S. Cl. CPC A61B 5/4803; G10L 25/66; G10L 25/30; A61B 5/7267; G10L 25/24; G06T 13/80; G10L 25/21

(57) ABSTRACT

A system for measuring neuromotor disorders from speech is configured to receive an audio recording that includes spoken speech and compute feature coefficients from at least a portion of the spoken speech in the audio recording. The feature coefficients represent at least one characteristic of the spoken speech in the audio recording. One or more vocal tract variables may be computed from the feature coefficients. The vocal tract variables may represent a physical configuration of a vocal tract associated with at least one of the one or more sounds. The vocal tract variables and/or the feature coefficients are used to determine if a disorder that affects neuromotor speech is present.

### STATEMENT REGARDING FEDERAL FUNDING

This invention was made with Government support under Grant No. FA8702-15-D-0001 awarded by the U.S. Air Force, and under Grant No. U.S. Pat. No. 1,514,544 awarded by the National Science Foundation. The Government has certain rights in the invention.

### BACKGROUND OF THE INVENTION

This invention relates to identification of disorders by analyzing speech patterns of a subject. Speech patterns can indicate the presence of certain disorders including psychological, neurotraumatic, neurodegenerative, and neurodevelopmental disorders. Using depression as an example, if a person is experiencing a depressive episode, their vocal source, vocal tract, and other motor control components of speech may form certain sounds differently than they otherwise would in the absence of depression. These sounds can indicate whether the subject is experiencing depression. This can be useful in making a diagnosis, especially if the subject is remote and only able to talk with a practitioner via telephone or video.

### SUMMARY OF THE INVENTION

A system that can identify neuromotor disorders from analyzing a patient's speech can increase accuracy, efficiency, and efficacy in making a diagnosis of the disorder. Changes in speech production that occur as a result of psychomotor slowing or other changes in a user's speech are difficult to detect without detailed analysis of the speech. Moreover, changes in a user's speech due to disorders such as depression can be subtle. Therefore, it can be difficult for a clinician to identify objectively whether a patient's speech indicates the presence of a disorder.

In addition, a system that identifies changes and problems in the way that a user's vocal tract articulates sound can improve detection of a neuromotor disorder and provide greater insight into the disorder. For example, depression can cause a person's vocal tract to produce sounds differently, e.g. to slow, slur, or produce less acoustic energy in particular ways or parts of speech. A system that can not only analyze the changes in the acoustic aspects of the speech, but can analyze the motion or position of elements of the vocal tract can provide a more accurate determination of the presence of a disorder such as depression.

In an embodiment, these and other advantages may be achieved, for example, by a method for measuring neuromotor coordination from speech. The method may include receiving an audio recording that includes spoken speech and computing feature coefficients from at least a portion of the spoken speech in the audio recording, the feature coefficients representing at least one characteristic of the at least a portion of the spoken speech in the audio recording. One or more vocal tract variables may be computed from the feature coefficients. The one or more vocal tract variables may represent a physical configuration of a vocal tract associated with at least one of the one or more sounds. The method may also include determining a measurement of a disorder based at least in part on a degree of correlation between two or more of the vocal tract variables.

One or more additional features may be included. For example, the feature coefficients may be cepstral coefficients which represent an audio power spectrum of the portion of the spoken speech, or may be formants. The vocal tract variables may be generated by a neural network, and the feature coefficients may be inputs to the neural network. The neural network may include stored parameters, which may include data from the Wisconsin X-Ray Microbeam database representing vocal tract variables associated with audio data.

The method may also include estimating a glottal state of the vocal tract, which may include performing acoustic measurements of the audio signal and/or providing the feature vectors to a neural network trained to estimate glottal vocal tract variables.

The method may also display an image of a vocal tract on a display device. The display device may be configured to play the audio recording and simultaneously animate the image of the vocal tract to display the physical configuration of the vocal tract of the speaker to provide visualization of where an articulatory deviation may occur due to a disorder.

The method may also associate the vocal tract variables with an utterance within the audio recording. Also, determining the measurement of the disorder may include correlating time-dependent functions of the at least one vocal trace variables. The time correlation dependent functions can include, for example, a channel-delay correlation matrix of the vocal tract variables and/or cepstral coefficients.

An eigenspectrum of the channel-delay correlation matrix can be generated. Magnitudes of eigenvalues within the eigenspectrum can indicate a disorder that affects speech. Thus, these magnitudes can be used in determining the measurement of the disorder.

Determining the measurement of the disorder may include computing changes in articulator kinematics as determined through phasing of coupled oscillatory models of articulatory gestures derived from vocal tract variables.

In another embodiment, a system for measuring neuromotor coordination from speech includes a processor configured to execute instructions stored on a non-transitory medium. The instructions may cause the processor to receive an audio recording that includes spoken speech; compute feature coefficients from at least a portion of the spoken speech in the audio recording, the feature coefficients representing at least one characteristic of the at least a portion off the spoken speech in the audio recording; compute, from the feature coefficients, one or more vocal tract variables representing a physical configuration of a vocal tract associated with at least one of the one or more sounds; and determine a measurement of a disorder based at least in part on a degree of correlation between two or more of the vocal tract variables.

The system may include one or more additional features. For example, the feature coefficients may be cepstral coefficients, which represent an audio power spectrum of the portion of the spoken speech, or may be formants that represent vocal tract resonances. The vocal tract variables may be generated by a neural network or other machine learning systems, and the feature coefficients may be inputs to the neural network or other machine learning systems. The neural network may include stored parameters, which may include data from the Wisconsin X-Ray Microbeam database representing vocal tract variables associated with audio data.

The method may also include estimating a glottal state of the vocal tract, which may include performing acoustic measurements of the audio signal and/or providing the feature vectors to a neural network trained to estimate glottal vocal tract variables.

The method may also display an image of a vocal tract on a display device. The display device may be configured to play the audio recording and simultaneously animate the image of the vocal tract to display the physical configuration of the vocal tract of the speaker.

The method may also associate the vocal tract variables with an utterance within the audio recording. Also, determining the measurement of the disorder may include correlating time-dependent functions of the at least one vocal trace variables. The time correlation dependent functions can include, for example, a channel-delay correlation matrix of the vocal tract variables and/or cepstral coefficients.

An eigenspectrum of the channel-delay correlation matrix can be generated. Magnitudes of eigenvalues within the eigenspectrum can indicate a disorder that affects speech. Thus, these magnitudes can be used in determining the measurement of the disorder.

Determining the measurement of the disorder may include computing changes in articulator kinematics as determined through phasing of coupled oscillatory models of articulatory gestures derived from vocal tract variables

Other features and advantages of the invention are apparent from the following description, and from the claims.

### BRIEF DESCRIPTION OF THE DRAWINGS

FIG. 1 is a block diagram illustrating an overview of a system for analyzing human speech to determine the presence of a disorder that affects neuromotor speech.

FIG. 2 is a block diagram of the system of FIG. 1.

FIG. 3 is a flowchart illustrating a process for analyzing human speech to determine the presence of a disorder that affects neuromotor speech.

FIG. 4 is an example display for presentation to a clinician.

Like reference numbers in the drawings depict like elements.

### DETAILED DESCRIPTION

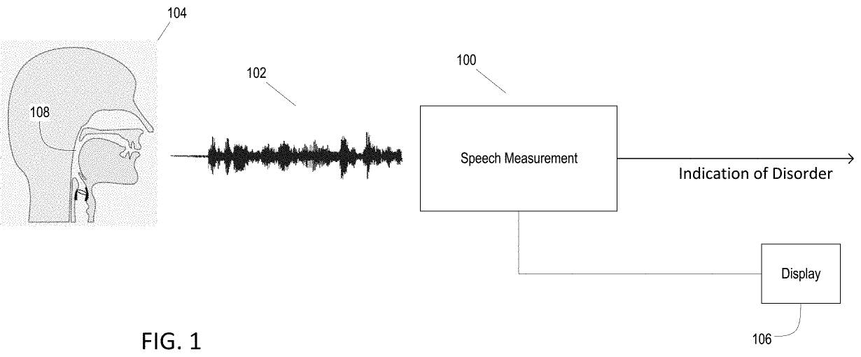

Referring to FIG. 1, a system 100 that may be used to process a speaker's 104 voice to identify underlying neuromotor and/or physiological conditions, for example my measuring the speaker's neuromotor control of speech. The system 100 is configured to receive a waveform 102 that represents speech from a user 104. Waveform 102 may include multiple channels (not shown). If the user 100 is suffering from a condition that affects his or her speech such as, for example, depression, the speech may include biomarkers that indicate the condition. These markers can include sounds or sound patterns within waveform 102 that provide information about the way the user's 104 vocal tract 108 is forming the sounds. As is known, there are many parts to the vocal tract that produce speech including the trachea, vocal and ventricular folds, epiglottis, buccal cavity, nasal cavity, tongue, lips, teeth, hard and soft palates, and the like. If a person has a speech disorder, some or all of these elements of the vocal tract may not operate normally and may produce sounds in potentially subtly abnormal ways. Similarly, if muscle control of the vocal tract is impaired by depression, intoxication, stroke, or other causes, sounds within the speech may be slurred or otherwise malformed. System 100 analyzes the waveform to determine if such biomarkers are present and, if so, can provide a determination as to whether the user 100 has a particular condition.

Conditions that affect the user's 104 speech include, but are not limited to depression, autism, attention deficit hyperactivity disorder, strokes, oral cancer, laryngeal cancer, Huntington's disease, dementia, amyotrophic lateral sclerosis (ALS), or other types of apraxia or dysarthria. In embodiments, based on biomarkers in the waveform 102, system 100 can detect a speech disorder and/or provide a determination as to the condition that may be causing the speech disorder. This document will use depression as an example. However, it should be appreciated that system 100 can be configured to detect and make a determination as to the presence or cause of any type of speech disorder, including but not limited to those listed above.

In one or more embodiments, in addition to making a determination as to the presence and source of a speech condition, system 100 may include or provide information to a display 106 that can provide visual information or animations of the user's 104 speech to a clinician. This may aid the clinician in assessing and treating the user's 104 disorder.

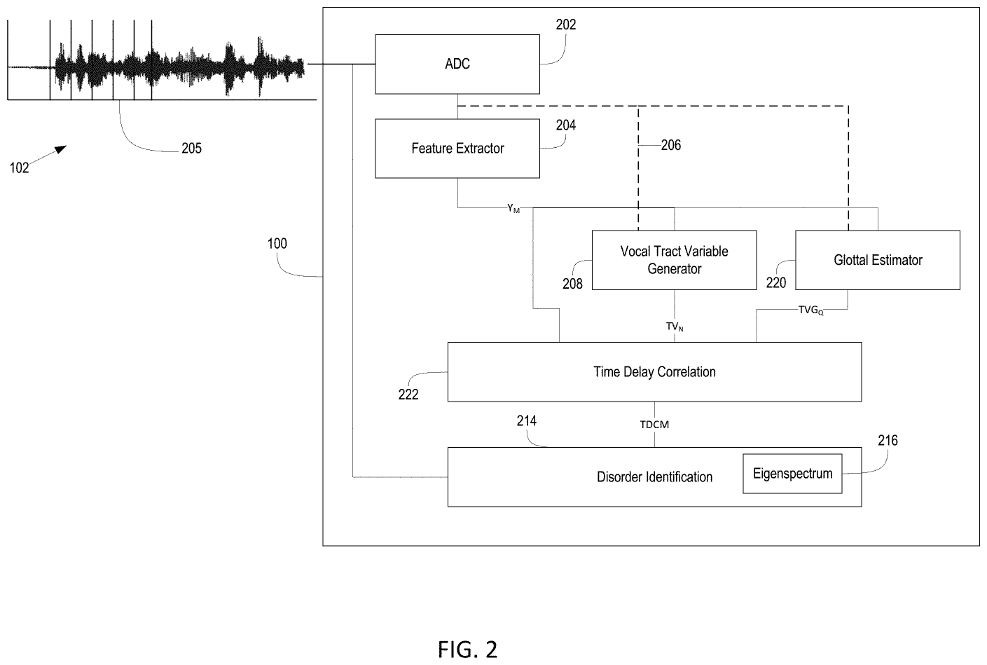

Referring to FIG. 2, system 100 is shown in greater detail. In at least some embodiments, system 100 may be implemented in software. For example, system 100 may comprise various software modules, libraries, and the like that may be stored on a non-transitory medium which, when executed by a processor, cause the processor and/or an associated computer system to perform the functions and implement the features described below. In other embodiments, system 100 may be implemented in hardware or circuitry designed to perform the functions and implement the features described below. In other embodiments, parts of system 100 may be implemented in software while other parts may be implemented in hardware. These software and hardware parts may operate together and communicate with each other, again, to perform the functions and implement the features described below.

The system 100 may include an audio input 201 that receives the audio waveform 102. Depending on the format of the waveform 102, the system 100 may optionally contain analog to digital converter (ADC) 202 to sample convert the waveform 102 into a digital waveform if, for example, waveform 102 is not provided in digital format or if waveform 102 needs to be resampled.

The system 100 may include a feature extractor 204 that receives the digital version of waveform 102 and produces feature coefficients Y_{M} representing characteristics of at least a portion of the acoustic waveform 102. For example, the feature coefficients Y_{M} may include formats, Mel-Frequency cepstral coefficients, log-frequency band energy coefficients, other acoustic energy coefficients, or a combination thereof.

The feature coefficients Y_{M} produced by the feature extractor 204 are numerical vectors that each correspond to time (e.g. a segment 205) of the waveform 102. In an example, the feature extractor 204 samples the waveform 102 at a sampling rate of 100 Hz and produces a sequence of feature coefficients Y for each M sample of the waveform 102. The elements of each feature vector are numerical representations of characteristics of the corresponding audio segment.

In an embodiment, the feature extractor 204 includes a short-time spectral analyzer that accepts the audio waveform 102, performs time windowing, Fourier analysis, and summation of energy over the ranges of the frequency bands.

The system 100 also includes a vocal tract variable generator 208 that receives the feature coefficients Y_{M} and produces vocal tract variable TV_{N} vectors. The vocal tract variables are numerical representations, specified in terms, for example representing a state of the user's 104 vocal tract 108 during articulation of the sound in the waveform 102 including, but not limited to, a state of the time-varying place (e.g. location along the oral cavity) and time varying manner (e.g. degree of constriction at the location) of characteristics of the position. For example, the vocal tract variables may include, but are not limited to, constriction degree and location of the lips, tongue tip, tongue body, velum, and glottis. Other vocal tract variables that can be included may describe features or positions of the nasal cavity, buccal cavity, nostrils, epiglottis, trachea, hard palate, or any other element of a person's vocal tract.

In embodiments, these TV vectors provide a way for the system to device biomarkers that are not constrained by the formant representation, but rather use the entire speech signal or a portion of the speech signal that is not directly mapped to the feature coefficient vectors. The vocal tract variable generator 208 may produce a TV vector for each sequence of feature coefficients Y that it receives, i.e. a TV vector for each sample of the waveform 102. But, additionally or alternatively, the vocal tract variable generator 208 generates a TV vector associated with a group of feature coefficients, i.e. a one-to-many mapping of TV vectors to feature coefficient vectors. For example, assuming that the user 104 articulated the word “No,” the feature extractor may produce a feature coefficient vector Y for each sampled segment 105 of the waveform. Thus, there may be a sequence of feature coefficient vectors Y_{N} associated with the “N” sound of the word “No,” and another sequence of feature coefficient vectors Y_{O} associated with the “O” sound of the word “No.” In an embodiment, the vocal tract variable generator 208 may also produce a TV vector that corresponds to the “N” sound (and represents the vocal tract position during utterance of the “N” sounds) from the sequence of feature coefficient vectors Y_{N}, and another TV vector that corresponds to the “O” sound (and represents the vocal tract position during utterance of the “O” sound) from the sequence of feature coefficient vectors Y_{O}.

In some embodiments, the vocal tract variable generator 208 may use samples from the waveform, in place of or in addition to the feature coefficient vectors Y_{M}, to generate the TV vectors, as indicated by dotted line 206.

The vocal tract variable generator may be implemented by a neural network. In embodiments, the neural network may be trained using a database of vocal tract training variables such as the Winsconsin X-Ray Microbeam (XRMB) database, which includes naturally spoken utterances along with XRMB cinematography of the mid-sagittal plane of the vocal tract with pellets placed at points along the vocal tract. In embodiments, the TV vectors include trajectory data (referred to as pellet trajectory) recorded for the individual articulators: e.g. Upper Lip, Lower Lip, Tongue Tip, Tongue Blade, Tongue Dorsum, Tongue Root, Lower Front Tooth (Mandible Incisor), Lower Back Tooth (Mandible Molar). These data may represent the way the articulators move during utterance as opposed to absolute position of the individual articulators. Because the physical X-Y positions of the pellets may be closely tied to the anatomy of the user 104, the pellet trajectories may provide relative measures of the articulators that reduce or remove dependence on the individual user's 104 anatomy. Thus, the TV vectors may specify the salient features of the vocal tract area function more directly than the pellet trajectories and are relatively speaker independent. In embodiments, the pellet trajectories are converted (by the vocal tract variable generator 208 or prior to training the vocal tract variable generator 208) to TV trajectories using geometric transformations.

The system 100 may optionally include a glottal estimator 220 which may receive the feature coefficient vectors Y_{M} and produce glottal vocal tract variable vectors TVG_{Q} that estimate articulation by or near the user's 104 glottis. This can be helpful to provide a more accurate model of the glottis if, for example, the audio waveform 102 was recorded without sensors placed near the user's 104 glottis. Glottal estimator 220 may use an aperiodicity, periodicity, and pitch detector that estimates the proportion of periodic energy and aperiodic energy in the speech signal 102 and/or the feature coefficient vectors Y along with the pitch period for the periodic component. In embodiments, glottal estimator 220 uses a time domain approach and is based on the distribution of the minima of the average magnitude difference function of the speech signal. If needed or desired, the glottal vocal tract variable vectors TVG_{Q} can be used in conjunction with the vocal tract variables TV_{M} to enhance the accuracy of glottal-related vocal tract variables. In some embodiments, the glottal estimator 220 may comprise a neural network or other machine learning module to produce the glottal vocal tract variable vectors.

As noted above, the system may correlate the vocal tract activity with the sounds in the speech waveform 102. This is useful because human speech may contain hysteresis (for example, the way a sound is physically formed by the vocal tract can depend on the way the previous sound was physically formed). It can also be useful in correlating the vocal tract activity with the original waveform 102 so that they can be animated and played back in a time synchronous manner. To correlate the time of the vocal tract activity with the waveform 102 system 100 may include a time delay correlation module 222 that receives the feature coefficient vectors Y, the waveform 102, and/or the vocal tract variable vectors TV and performs a time delay correlation.

For each speech signal 102, the time delay correlation module 222 generates a channel delay correlation matrix TDCM from the TV vectors and/or the feature coefficients Y using a time-delay embedding at a constant delay scale. For example, if the sample rate was 100 Hz, a delay scale of 7 samples would introduce delays into the signals in 70 ms increments. The time delay correlation matrix provides information about the mechanisms underlying the coordination level. Each time delay correlation matrix may have a dimensionality of (MN*NM), where M is the number of channels and N is the time delay per channel.

The system 100 also includes a disorder identification module 214 that processes the TDCM to determine whether a disorder is present. The disorder identification module 214 may generates a rank ordered eigenspectrum 216 from the TDCM. In embodiments, the eigenspectrum may be an MN-dimensional feature vector. The eigenvalues in the spectrum may be ranked in order of magnitude (e.g. the rank 1 eigenvalue is the largest and the rank MN eigenvalue is the smallest, for example).

The disorder identification module may process the eigenspectra to determine a degree of correlation between two or more of the vocal tract variables. This degree of correlation may represent correlation of phase, rise time, fall time, slope, or other time-based characteristics of the vocal tract variables, and/or may include correlation of amplitude, peak-to-peak values, or other magnitude-based characteristics of the vocal tract variables. The degree of correlation between vocal tract variables can indicate the presence of a speech irregularity that may be caused by a neuromotor disorder. In embodiments, the disorder identification module may include functions that measure the degree of correlation between vocal tract variables by processing the vocal tract variables directly or by processing the eigenspectrum.

The eigenvalues may, in an embodiment, be proportional to the amount of correlation in the direction of their associated eigenvectors and can be used to identify a disorder. For example, depressed speech has few eigenvalues with significant magnitudes. Therefore, depressed speech can be identified by evaluating the eigenspectrum to determine if the recorded speech that generated the eigenspectrum includes the markers for depressed speech. One of skill in the art will recognize that depressed speech is used merely as an example, and that the eigenspectrum can be evaluated by the disorder identification module 204 to determine if the recorded speech matches markers for other types of disorders. The use of eigenspectra is only one of many ways to represent a change in vocal tract variable dynamics. For example, based on vocal variables, one can estimate the phasing relation across articulatory gestures as determined by a custom implementation of coupled oscillator planning, and the associated Task Dynamics model of speech motor control to generate relevant speech kinematics. See, for example, A. C. Lammert et al., A Coupled Oscillator Planning Model Account of the Speech Articulatory Coordination Metric With Applications to Disordered Speech, 12th International Seminar on Speech Production, which is incorporated here by reference in its entirety.

Yet another example of an approach to measure changes in vocal tract variable dynamics involves entropy measures of system dynamics.

In embodiments, disorder identification module 204 may be implemented as a neural network. It can be trained with model eigenvalues that identify a particular disorder, such as depression. One skilled in the art will recognize that neural networks and training models for identifying a disorder from vocal characterization may be complex. They may require not only the position of articulatory features of the elements of the vocal tract, but transitory movements of those elements as they transition from sound to sound. For example, the previous sound and position of the vocal tract elements may affect articulation of the next sound and/or the positions that the vocal tract elements go through to get to the next sounds. The model may need to include such information so that the system can provide accurate physical configuration of the vocal tract over time.

In general, as described above, the system 100 is configured to analyze the speech of a user 104 and make a determination, based on the speech, as to whether a disorder is present. The system 100 can use feature coefficients Y representing qualities of the audio recording, TV vectors representing articulation of the user's 104 vocal tract, or both in the analysis to determine if a disorder is present. This provides advantages in that inclusion of TV vectors, for example, produces more accurate determination of whether a disorder is present. Also, information about articulation of the vocal tract can be presented to a clinician for further analysis.

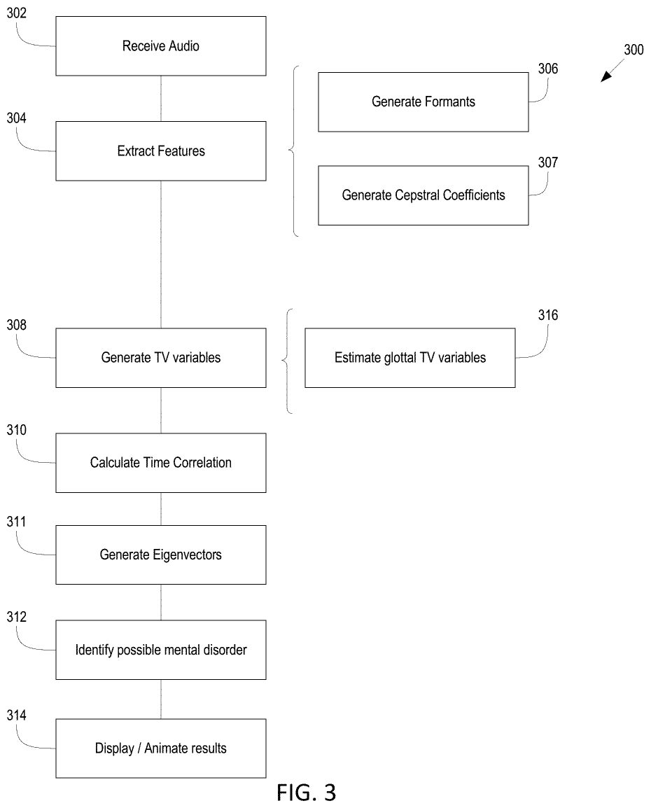

Referring to FIG. 3, flowchart 300 displays in general terms the process that the system 100 implements to analyze recorded speech to determine if a disorder is present. The process may be implemented, at least in part, by the system 100 described above.

In box 302, the system 100 may receive an audio recording (e.g. waveform 102) having one or more channels that include speech spoken by a user 14. In box 304, the system 100 may sample and extract audio features from the audio recording. Extracting the audio features may include generating formants (box 306), generating cepstral coefficients (box 307), or generating other variables that represent the audio within the recording.

In box 308, the system 100 may generate TV vectors that represent articulation and position of elements of the speaker's vocal tract. These variables may indicate the position and/or relative position or movement of articulatory vocal elements such as the lips, teeth, vocal folds, etc. In some embodiments, the system 100 may estimate TV vectors (box 316) related to glottal articulatory elements in the vocal tract.

In box 310, the system 100 may generate a time correlation matrix that provides time and delay information in relation to the TV vectors. The time correlation matrix may be useful in capturing temporal information related to the dynamics of the articulation of the vocal tract. In box 311, the system 100 may generate an eigenspectrum having eigenvectors that represent the user's 104 speech and can be used to identify, from the speech, whether a disorder is present. In box 312, the system may determine whether the speech indicates the presence of a possible mental disorder.

In box 314, the system 100 may display its findings regarding the presence of a mental disorder to a clinician. The system 100 may also provide an animation displaying the operation of the user's 104 vocal tract as the audio recording 102 is played.

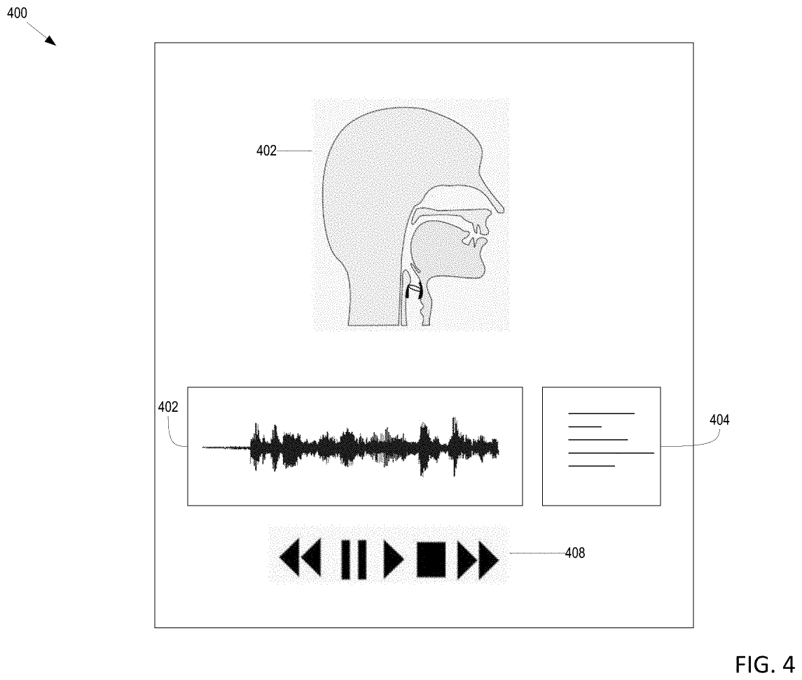

Referring to FIG. 4, an example display 400 may be presented to a clinician to evaluate the user's 104 speech. The display 400 may be displayed on a computer monitor, television, or other device that can present the display with video and/or audio. The display 400 may include an animation 402 of the vocal tract that shows how elements of the vocal tract move while the audio recording 102 is played, an image 404 of the recorded waveform, one or more panels 406 displaying information about the user 104 and the analyzed speech, and controls 408 that the clinician can use to control the display 400. In embodiments, the display 400 may highlight portions of the vocal tract animation that may indicate the presence of a disorder while the animation is running. For example, the animation may show decline in coordination of articulators as determined through eigenspectral analysis or through changes in kinematics as determined through phasing of coupled oscillatory models of articulatory gestures.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

1. A method for measuring neuromotor coordination from speech:
receiving an audio recording that includes spoken speech;
computing time varying feature coefficients from at least a portion of the spoken speech in the audio recording, the feature coefficients representing at least one characteristic of the at least a portion off the spoken speech in the audio recording;
computing, from the feature coefficients, one or more time varying vocal tract variables representing time variation of physical configuration of a vocal tract, the time varying vocal tract variables associated with at least one of the one or more sounds; and
determining a measurement of a disorder based at least in part on a degree of correlation between at least two of the vocal tract variables.

2. The method of claim 1 wherein the feature coefficients represent characteristics of an audio power spectrum of the portion of the speech.

3. The method of claim 2 wherein the feature coefficients comprise cepstral coefficients.

4. The method of claim 1 wherein computing the vocal tract variables comprises providing the feature coefficients as inputs to a neural network, and using the neural network to compute the vocal tract variables from the feature coefficients.

5. The method of claim 4 wherein the neural network comprises stored parameters determined using the Wisconsin X-Ray Microbeam database representing vocal tract variables associated with audio data.

6. The method of claim 1 wherein computing the vocal tract variables includes estimating a glottal state.

7. The method of claim 6 wherein estimating the glottal state comprises calculating the glottal state from acoustic measurements of the audio signal.

8. The method of claim 1 further comprising displaying an image of a vocal tract on a display device.

9. The method of claim 8 further comprising playing the audio recording and simultaneously animating the image of the vocal tract to display the constriction location and degree of articulators along the vocal tract of the speaker from the cepstral coefficients.

10. The method of claim 1 further comprising associating the vocal tract variables with an utterance within the audio recording.

11. The method of claim 1 wherein determining the measurement of the disorder comprises computing time correlation dependent functions of the at least one vocal tract variables.

12. The method of claim 11 wherein computing the time correlation dependent functions comprises generating a channel-delay correlation matrix of the vocal tract variables and/or cepstral coefficients.

13. The method of claim 12 further comprising generating an eigenspectrum of the channel-delay correlation matrix, and determining the measurement of the disorder comprises identifying eigenvalues within the eigenspectrum that have magnitudes indicating depressed speech.

14. The method of claim 1 wherein determining the measurement of the disorder comprises computing changes in articulator kinematics as determined through phasing of coupled oscillatory models of articulatory gestures derived from vocal tract variables.

15. The method of claim 1 wherein determining the measurement of the disorder further includes a time delay correlation of the vocal tract variables.

16. A system for measuring neuromotor coordination from speech, the system comprising:
a receiver to receive an audio recording that includes spoken speech;
a feature extractor configured compute feature coefficients from at least a portion of the spoken speech in the audio recording, the feature coefficients representing at least one characteristic of the at least a portion off the spoken speech in the audio recording;
a vocal tract variable generator configured to generate one or more vocal tract variables representing a physical configuration of a vocal tract associated with at least one of the one or more sounds; and
a disorder identification module configured to determine a measurement of a disorder based at least in part on a degree of correlation between at least two of the vocal tract variables.

17. The system of claim 16 wherein the cepstral coefficients represent an audio power spectrum of the portion of the spoken speech.

18. The system of claim 17 wherein the feature coefficients are cepstral coefficients.

19. The system of claim 16 the vocal tract generator comprises a neural network that computes the vocal tract variables from the feature coefficients.

20. The system of claim 19 wherein the neural network comprises stored parameters using the Wisconsin X-Ray Microbeam database representing vocal tract variables associated with audio data.

21. The system of claim 16 further comprising a glottal estimator configured to generate vocal tract variables includes estimating a glottal state.

22. The system of claim 16 further comprising a display interface configured to display an image of a vocal tract.

23. The system of claim 22 wherein the display interface is configured to play the audio recording and simultaneously animate the image of the vocal tract to display the constriction location and degree of articulators along the vocal tract of the speaker from the cepstral coefficients.

24. The system of claim 16 further comprising a time delay correlation module that associates the vocal tract variables with an utterance within the audio recording.

25. The system of claim 16 further comprises a time delay correlation module that computes time correlation dependent functions of the at least one vocal trace variables.

26. The system of claim 25 wherein the time delay correlation module computes the time correlation dependent functions by generating a channel-delay correlation matrix of the vocal tract variables and/or cepstral coefficients.

27. The system of claim 26 further the time delay correlation module generates an eigenspectrum of the channel-delay correlation matrix; and
the disorder identification module determining the measurement of the disorder comprises identifying eigenvalues within the eigenspectrum that have magnitudes indicating depressed speech.

28. The system of claim 16 wherein determining the measurement of the disorder comprises computing changes in articulator kinematics as determined through phasing of coupled oscillatory models of articulatory gestures derived from vocal tract variables.
